SEQUENCE LISTING (10 sequences)

SEQ ID NO: 1 - DNA, 21 bp, Artificial Sequence
  oligonucleotide primer
agctgtgctcgacgttgtcac

SEQ ID NO: 2 - DNA, 20 bp, Artificial Sequence
  oligonucleotide primer
aatcgggagcggcgataccg

SEQ ID NO: 3 - DNA, 21 bp, Artificial Sequence
  oligonucleotide primer
cgtggtgatgtggagtattgc

SEQ ID NO: 4 - DNA, 19 bp, Artificial Sequence
  oligonucleotide primer
ttgcagcagaaaagccgcc

SEQ ID NO: 5 - DNA, 21 bp, Artificial Sequence
  oligonucleotide primer
ggaaagcttcggatttggagc

SEQ ID NO: 6 - DNA, 32 bp, Artificial Sequence
  oligonucleotide primer
cggctgcagcgtctgaaataaaacaatagaac

SEQ ID NO: 7 - DNA, 22 bp, Artificial Sequence
  oligonucleotide primer
tgaggatcctttcgcatgattg

SEQ ID NO: 8 - DNA, 20 bp, Artificial Sequence
  oligonucleotide primer
ttggtaccccagagtcccgc

SEQ ID NO: 9 - DNA, 25 bp, Artificial Sequence
  oligonucleotide primer
tgtggcatttattgaaatggcactg

SEQ ID NO: 10 - DNA, 29 bp, Artificial Sequence
  oligonucleotide primer
ctatatctagacatcgtaattttaagacg

(19) United States
(12) Patent Application Publication
(10) Pub. No.: US 2003/0115641 A1
(43) Pub. Date: Jun. 19, 2003

(54) Transformation of plants by electroporation of cultured explants

(76) Inventors: Michael S. Dobres, Philadelphia, PA (US); Aidyn Mouradov, Koln (DE); Hong Zhang, Upper Darby, PA (US)

Correspondence Address: Woodcock Washburn Kurtz, MacKiewicz & Norris LLP, One Liberty Place - 46th Floor, Philadelphia, PA, 19103, US

(21) Appl. No.: 09/911,588

(22) Filed: Jul. 24, 2001

(51) Int. Cl. A01H 1/00

(52) U.S. Cl. 800/292

(57) ABSTRACT

The present invention provides methods of transforming plants using electroporation of explants, and methods of producing transgenic plants via electroporation of explants. The methods of the invention are also useful for generating transgenic plants free of marker genes. Also provided are plants produced by the methods of the invention.

### TECHNICAL FIELD OF THE INVENTION

The present invention relates generally to the fields of plant cellular and molecular biology. More particularly, the invention relates to methods of electroporation of cultured explants and uses of the methods to produce transgenic plants.

### BACKGROUND OF THE INVENTION

There are several alternative methods available for the transformation of plant tissues. They have been widely described in the literature, and are reviewed by Weising et al., Annu. Rev. Genet., 22, 421 (1988). They include, but are not limited to the methods described below. Agrobacterium mediated transformation is a method whereby the desired trait gene is first placed between the T-DNA border regions of a T-DNA plasmid. The T-DNA plasmid is then introduced into a suitable Agrobacterium strain. The resulting Agrobacterium cells have the ability to transfer DNA located between the T-DNA borders to plant cells. See, e.g., U.S. Pat. Nos. 4,940,838, 5,149,645, 5,464,763, and 6,051,757.

Others have reported the use of the gene gun to introduce DNA into plant cells. See, e.g., U.S. Pat. Nos. 4,945,090 and 5,036,006. This method requires that the gene of interest first be immobilized on small metal particles. The particles are then fired into plant cells. Once in the plant cells, the DNA may solubilize and become integrated into the plant genome.

Another method involves immobilization of DNA on silicon fibers. The fibers are then vortexed in the presences of plant cells. The resulting mechanical disruption allows the fibers to pierce the cells and deposit DNA into the cells. See, e.g., U.S. Pat. No. 5,3030,523.

Still others have used microinjection of DNA into plant cells (U.S. Pat. No. 4,743,548); non-pulsed continuous electric fields (U.S. Pat. No. 5,371,003); polycationic liposomes (U.S. Pat. No. 5,286,634); and magnetophoretic delivery (U.S. Pat. No. 5,516,670).

Electroporation is a method by which pulses of electricity are used to facilitate the entry of DNA and other molecules into living cells. It is believed that the pulses transiently cause the formation of pores in the plasma membrane large enough to allow the entry of DNA molecules through the membrane into the cell (see Shillito, Molecular Improvement of Cereal Crops, pp. 9-20, I. K. Vasil ed. (1999)). For plants, the cell wall represents an additional barrier through which DNA molecules must pass. For this reason, early studies on the electroporation of DNA into plant cells entailed the complete enzymatic removal of the cell wall. Such “cell-wall free” plant cells are known as “protoplasts.”

Electroporation of plant protoplasts has been reported in the literature for many plant species. Examples include dicot species such as tobacco (Shillito et al., Bio/technology, 3:1099-1103 (1985)), soybean (Christou and Swain, Theoretical and Applied Genetics, 79, 337 (1990)) and sugar beet (Lindsey and Jones, Plant Molecular Biol. 10, 43 (1987)), as well as monocot species such as rice (Tada et al., Theor. Appl. Genet 80, 475 (1990)), and corn (Fromm et al., Nature, 319,791 (1986)).

Electroporation of plant protoplasts (i.e., cells from which the cell wall has been completely removed by enzymatic digestion) is described in, e.g., U.S. Pat. Nos. 5,231,019, 4,684,611, and 5,508,184. The usefulness of this method is limited by the difficulty encountered in the regeneration of whole fertile transgenic plants from transformed protoplasts. An alternative approach disclosed in U.S. Pat. No. 5,629,183 is to electroporate DNA in pre-germinated pollen cells. Transformed pollen cells are then use to fertilize the ova of a plant.

Suspension cultures are liquid cell cultures in which loose cell aggregates are maintained as a fine suspension of cells. Such cultures represent a reasonable target for electroporation since the increased cell surface area likely increases the opportunity for DNA uptake. U.S. Pat. No. 5,679,558 discloses a method which avoids the use of plant cell protoplasts, but requires the preparation of embryogenic suspension cultures as a target for electroporation. The preparation of such cultures is particularly time consuming, and regeneration of plants from such cultures is not always possible. It requires that rice seed derived material be cultured on solid plant growth media for 4 weeks. This callus material is then transferred to liquid media and sub-cultured weekly for a period of two months prior to the first electroporation (see Examples 1 and 2 thereof).

To avoid the use of protoplasts or suspension cultures, D'Halluin et al, reported the production of transgenic maize by electroporation of enzymatically treated zygotic embryos (D'Halluin et al., Plant Cell, 4, 1495 (1992)). Zygotic embryos represent “true” plant embryos that can be derived from seeds prior to or after seed dormancy has been established. This method provides for increasing the permeability of cell walls by partial enzymatic degradation. Guerel and Gozukirmizi reported a modification of this method for barley zygotic embryos in which enzymatically treated embryos were briefly cultured on plant growth media prior to electroporation. Guerel and Gozukirmizi, Plant Cell Reports, 19, 787 (2000). According to D'Halluin et al., the efficiency of this method is highly dependent on the quality of the immature embryos. Embryo quality was highest in spring. Reliance on seasonal factors imposes a barrier to the commercial use of embryos for electroporation.

Several studies have attempted to electroporate seed derived embryos (zygotic embryos) without the use of enzymatic digestion. Sorokin et al. (Plant Science 156: 227 (2000)) reported the production of fertile transgenic wheat plants using electroporation of intact wheat immature embryos. The transformation frequency obtained using this method was extremely low. From 1080 embryos electroporated only three transgenic plants were obtained. This corresponds to a transformation frequency of 0.28%. The low transformation frequency is presumably due to both the lack of an enzyme treatment and the seasonal variation in embryo quality reported by D'Halluin et al., Plant Cell, 4, 1495 (1992). Furthermore, the inability of this strategy to select for individually transformed cells may explain the low level of transformants obtained. Since no organogenic step was used to select transgenic plants, it is likely that the plants obtained were chimaeric, composed of a mixture of transformed and non-transformed cells.

Suspensions of mesophyll cells with intact cell walls can be obtained from the leaves of many plant species by mild digestion with a cell wall degrading enzyme. The partial digestion and the increased surface area of such cell suspensions may increase the ability to uptake DNA. However, regeneration of whole plants from such enzymatically treated cell suspensions is difficult, and is not suitable as part of a general transformation method. The transient electroporation of Tobacco Mosaic Virus (TMV) RNA into enzymatically treated mesophyll cell suspensions of tobacco was demonstrated by Morikawa et al (Gene 41:121-124 (1986)). The high infectivity of RNA makes estimates of the effectiveness and suitability of this treatment for plasmid DNA difficult. Though data was presented that the TMV RNA was capable of replicating and forming infective particles, no evidence of transgenic plants derived from the infected cells was presented. Indeed, it would not be expected that such freely replicating virus particles would integrate into the plant genome.

Several investigators have reported the use of specific chemicals to increase the uptake of DNA during electroporation. Chowrira et al. (Molecular Biotechnology, 3:17,1995) reported the use of lipofectin to facilitate the uptake of DNA during electroporation into intact nodal meristems. Buds on nodal segments were allowed to grow into whole plants and seeds were collected from them. The authors claimed that expression was seen in the seeds of transformed plants but no data was presented.

Songstad et al. (Plant Cell Tissue and Organ Culture, 33:195-201) reported the use of 0.2 mM spermidine during electroporation to introduce and obtain transient expression of GUS and anthocyanin constructs into cultured immature zygotic embryos of corn. No whole transgenic plants were obtained.

Akella et al (Plant Cell Reports (1993) 12: 1) reported the electroporation of cowpea in the presence of 2 mM spermidine, but no evidence of stable transformation and heritability was demonstrated.

Dekeyser et al.(The Plant Cell, 2: 591-602, 1990) reported the use of 0.2 mm spermidine to obtain transient expression of GUS in rice leaf bases. No permanent or stable transformation was obtained and no transgenic plants were obtained.

Somatic plant embryos can be derived from many plant species through the use of a lengthy in vitro culturing process. The successful production and germination of such embryos is often difficult to obtain. For example, Luong et al. reported transient gene expression in cassava somatic embryos (Luong et al., Plant Science 107, 105 (1995). No stable transformants were reported and the embryos were derived from in vitro cultures that were subcultured every 30 days.

U.S. Pat. No. 5,859,327 discloses methods for electroporation of intact tissue using short electrical pulses of less than 20 milliseconds. It does not disclose evidence for the transient or stable transformation of such cells or creation of transgenic plants from such tissue.

Several U.S. Patents disclose methods of electroporation of partially degraded monocot cells issued. For example, U.S. Pat. Nos. 5,384,253 and 5,472,869 describe a method for the electroporation of Zea mays suspension cells after enzymatic treatment; U.S. Pat. Nos. 5,641,664, 5,712,135, and 6,002,070 disclose the electroporation of enzymatically treated zygotic embryos of corn, the electroporation of wounded type I callus, and a method for electroporating seed derived rice tissues after an enyzmatic treatment.

The art is therefore in need of a method of transformation of plant cells which results in higher efficiency in the production of transgenic plants.

### SUMMARY OF THE INVENTION

The method of the invention overcomes the disadvantages of the methods in the art by avoiding reliance on seasonal variation, chemical treatment, and enzymatic digestion. The method of the invention uses cultured explants which are capable of undergoing organogenesis, allowing for selection and regeneration of true transgenic plants derived from single transformed cells.

The general procedure of the method of the invention is an efficient process for producing transgenic plants by subjecting cultured plant explants to long electrical pulses. More importantly, when marker genes and trait genes are transferred as separate molecules, the high efficiency of the method allows one to readily select for marker-free transgenic plants. Such marker-free transgenic plants are of great commercial value.

The method of the invention also allows the integration of a desired gene without neighboring plasmid DNA sequences, as well as the cotransformation of multiple genes. These properties have great commercial value because the first reduces the chance for transferring unwanted genetic sequences into host plants, and the second increases the ease with which traits can be stacked.

The method of the invention is applicable to any plant for which a tissue culture system is available or can be developed. The advantage of the culturing step is that it allows the selection of developmental stages best suited to withstand the electroporation process and subsequently allows the efficient and rapid regeneration of transgenic plants.

### DETAILED DESCRIPTION OF THE INVENTION

The method of the invention is directed to an efficient process for producing transgenic plants by subjecting cultured plant explants to electroporation using long electrical pulses.

Definitions

Various definitions are made throughout this document. Most words have the meaning that would be attributed to those words by one skilled in the art. Words specifically defined either below or elsewhere in this document have the meaning provided in the context of the present invention as a whole and as are typically understood by those skilled in the art.

As used herein, “intact” cells are cells that have not been subjected to enzymatic digestion, or partial enzymatic digestion, of their cell walls.

As used herein, “untreated” means that, prior to electroporation, plant tissue was not incubated or pre-incubated with spermidine, lipofectin, dimethyl sulfoxide, or any other polyamine, lipophilic, or hydrophobic agent or solvent the use of which is intended to increase the permeability of the plant cell wall or plant cell membrane to nucleic acids.

As used herein, “explant” refers to plant tissue that is directly excised from an intact plant, such as a leaf, petal, sepal, stamen, filament anther, root, or stem.

As used herein, “cultured” explants are explants which are cultured prior to electroporation on plant growth media containing plant growth regulators. The precise media composition on which a given plant species is cultured is known to those skilled in the art to be particularly species-dependent. For most commercially valuable species the general culture media have been widely described in the literature. As those skilled in the art would recognize, the general nutritional and growth requirements of cultured plant cells must be satisfied. The general nutritional, hormonal, and growth requirements of plant cells are well known, and a number of conventional culture media and growth protocols have been developed which satisfy these needs (hereinafter referred to as “nutritive medium” or “nutritive media”). See, e.g., MSG medium (Becwar, M. R., et al., “Developmental and Characterization of In Vitro Embryogenic Systems in Conifers” in Somatic Cell Genetics of Woody Plants, Ahuja, Kluwer, eds., Academic Publishing. Dordrecht, The Netherlands (1988)), Shenk-Hilderbrandt (SH) culture medium (Shenk et al, Can J. Bot., Vol. 50, pp. 199-204 (1972)), Murashige-Skoog (MS) Basal media (Murashige et al., Physiol Plant., Vol 15, pp. 473-97 (1962)), and White's medium (White, The Cultivation of Animal and Plant Cells, 2^{nd} ed., Ronald Press Co., New York (1963)). A Comprehensive list of plant culture media and culture protocol are found in Huang et al., Plant Tissue Culture Media, TCA Manual, Vol. 3 pp. 539-48, Tissue Culture Association, Rockville, Md. (1977). The disclosures of each of these references are incorporated herein by reference in their entirety. The foregoing culture media and culture protocols, as well as others known to those skilled in the art, can be employed in conjunction with the methods and media of the present invention. In accordance with the invention, however, sucrose, or other carbon sources including and not limited to glucose, sorbitol, mannitol, fructose and galactose, must necessarily be present, as should a source of vitamins such as Gamborgs B5 vitamins (B5) (Gamborg et al., Nutrient Requirements of Suspension Cultures of Soybean Root Cells Exp. Cells Res. 1968, 50, 151-158), and certain growth hormones such as, but not limited to, indoleacetic acid (IAA) and 2,4-dichlorophenoxyacetic acid (2,4,-D) and benzyladenine (BA). Suitable alternatives are known in the art.

Within this general framework, and in addition to the aforementioned components 2,4,-D and IAA and BA, the culture medium used in the current invention further comprises a nutritive medium. Preferably the medium is Murashige and Skoog (MS) medium though other media, such as those described above may, depending on the genus, be used.

The culture process renders the material more resistant to the rigors of electroporation and aids in the subsequent regeneration of transformed cells after electroporation. The culture period can be brief (as short as, for example, 5 days or less) or can be long (several months to several years). In either case it is important that the cultured explant still be capable of regeneration into a whole plant. In general we have found that, the brevity of the culture period increases regeneration frequency and minimizes the potential for mutations occurring during culture. It also makes this method well suited to commercial applications.

As used herein, a “long pulse” of electroporation means a pulse of at least about 21 milliseconds. As used herein, a “transgene” is a desired DNA to be electroporated into an explant resulting in a transgenic plant. Such transgenes include, but are not limited to, genes for disease resistance, insect resistance, virus resistance, fragrance biosynthesis, modified flowering time, modified flower shape and flower organ number, growth rate, increased or decreased plant height, increased or decreased branching, drought tolerance, altered metabolism (such as carbohydrate biosynthesis, terpene biosynthesis and nitrogen fixation) and altered photosynthetic capacity. Proof that the cells have taken up DNA is obtained by measuring the activity of a reporter gene, selecting transformed cells on selective media, and polymerase chain reaction (PCR) detection of transferred DNA. Additional or alternative proof can be obtained by DNA blot hybridization analysis (Southern or Dot Blot), as well as by segregation analysis of transformants.

Embodiments of the Invention

In one embodiment, the present invention is directed to a method of plant cell transformation using preculturing of plant explants on nutritive media followed by long pulse electroporation, then selection and generation of transformed plants.

In a preferred embodiment, an explant is cultured in an appropriate nutritive medium, with appropriate sources of vitamins, prior to electroporation with the desired transgene DNA (the “transgene”).

In one embodiment, the present invention is directed to a method of plant cell transformation that uses a selectable marker to be transfected along with the transgene. It is possible to eliminate the use of a selectable marker and screen plants based purely on the presence or absence of a reporter gene or trait gene. Both could be selected by convenient enzymatic methods. Reporter genes suitable for screening include, but are not limited to, the E. coli β-glucuronidase (GUS) gene (Jefferson et al., EMBO J., 6, 3901 (1987)), anthocyanin biosynthesis genes (McElroy et al., Trends Biotechnol 12, 62 (1984)), green fluorescent protein (Chalfie et al., Science, 263, 802 (1994)) or the luciferase gene from firefly Photinus pyramis (Ow et al., Science, 234, 856 (1986)).

When using a selectable marker, the particular selectable marked used would depend on the plant species, and commercial and regulatory factors. Suitable genes are well known in the art and include among others genes that encode disease resistance, insect resistance, color, fragrance, plant height and herbicide resistance. Other selectable markers have been widely described in the literature, and are reviewed by Weising et al., Annu. Rev. Genet., 22, 421 (1988). They include, but are not limited to, neomycinphosphotransferase (NPTII) (Bevan et al., Nature, 304, 185 (1983)), glyphosate resistance (Comai et al., Nature, 317, 741 (1985) and hygromycin (Van den Elzen et al., Plant Mol. Biol, 5, 299 (1985)).

In a preferred embodiment, the cultured explant is subjected to electroporation using a pulse lasting at least preferably about 21 milliseconds, more preferably about 60-400 milliseconds, more preferably about 70-300 milliseconds, more preferably 80-250 milliseconds, more preferably about 90-200 milliseconds, even more preferably about 90-150 milliseconds, and most preferably about 90-125 milliseconds. Pulses in the range of about 200-600 milliseconds may also be preferred, depending on the species of explant. In certain cases it is expected that very long pulses of 600-2000 milliseconds may be necessary. In such circumstances viability may be increased by decreasing the voltage to less than 100 volts. The voltage of electroporation is preferably in the range of about 50 to 200 volts. Determining the optimal length and voltage of the pulse may be accomplished by assays such as those described herein. In certain cases it is envisaged that pulses of less than 50 or higher than 200 volts may be necessary to successfully introduce DNA in plant cells. In such cases, it is likely that one may have to increase the pulse time to more than 500 milliseconds for pulses of less than 50 volts or decrease the pulse time to less than 20 milliseconds for pulses of more than 200 volts. In a similar way, it may also be necessary to modify the capacitance used in combination with very low or high pulse lengths and very low or high voltages. In either case, it would not involve undue experimentation for one skilled in the art to determine the appropriate combination of pulse time, voltage, and capacitance needed, in accordance with the disclosure herein.

In a preferred embodiment, the explant is subjected to electroporation at 100 volts and a pulse time of about 190 milliseconds. Generally, low voltage with a moderate pulse time (100-200 msecs) is optimal. A pulse time which is too long results in damage and death of tissue. A pulse time which is too short provides inadequate time for DNA to enter cells. The exact capacitance needed to produce the desired pulse time will vary depending on the exact conditions in the electroporation final buffer and callus mixture. It is believed that different tissues secrete certain conducting and/or chelating agents into the medium that alter the conductivity of the buffer. Thus, it is important to adjust the capacitance in trial experiments to obtain a suitable pulse time.

In one embodiment, the explant tissue is removed from the electroporation buffer immediately following electroporation, and the explant is placed on non-selective media to allow the plant tissue to recover and to allow for expression of the selectable marker gene. The explant is then placed on selective media to allow the growth of, and to reveal, those plants containing the selectable marker. The resulting plants are then screened for the presence of the selectable marker and/or the transgene, the presence of either of which indicates a successful transformation and production of a transgenic plant.

In another preferred embodiment, the method of the invention is used to generate transgenic plants which do not have selectable markers. The production of transgenic plants without markers is of extraordinary commercial value. There exists wide public, governmental, and regulatory opposition to the use of antibiotic genes and herbicide resistance genes in transgenic plants. A great deal of effort has, and is being, expended to develop “marker” free transgenic plants. These include methods for the excision of selectable markers using recombinases (Onouchi et al., Nucl. Acids Res. 19, 6373 (1991); Onouchi et al., Mol. Gen. Genet. 247: 653 (1995); and Ebinuma et al., In Vitro Cell. Dev. Biol. 37,103 (2001)), and the use of transposons (Gleave et al., Plant Mol. Biol., 40, 223 (1999)).

Plants which survive growth on selective media may be shown to lack the gene conferring such resistance, as shown in Example 1 below. Such plants are known as “escapes” which escape selection by kanamycin and are ordinarily presumed to be normal wild-type plants. However, as also shown in Example 1, sometimes the escape plants do in fact contain the transgene. One explanation for these escape transgenic plants is that they are derived from cells that escaped selection due to transient expression of the resistance gene. Transient expression occurs when a gene does not integrate into the chromosome of a plant, but is nonetheless recognized by the cells transcription and translation machinery. Transient expression typically occurs for two to seven days at levels detectable by biochemical assays. Cells that take-up both a resistance gene and a transgene may transiently express the resistance gene and yet incorporate the transgene. Transient expression of the resistance gene may confer a level of resistance suitable for the formation of a plant shoot. Once formed, such shoots (even in the absence of resistance expression), often escape selection, based purely on their size.

One may enhance the number of marker-free transgenic plants by reducing the intensity of selection, and by screening at a relatively earlier time point for marker-free transgenic plants. Thus, instead of placing kanamycin resistant shoots through a second selection step on an antibiotic-containing rooting media, shoots are transferred to rooting media without the antibiotic. This would be expected to increase the number of escapes, and increase the number of marker-free transgenic plants.

The ability to generate the relatively rare events that give rise to marker-free transgenic plants is due to the high transformation rate of the efficient method of the invention. With this method, cells are exposed to a homogeneous solution of DNA molecules. Unlike other electroporation methods, spermidine is not used. Spermidine is known to cause clumping and aggregation of DNA molecules. The use of moderately long pulses facilitates the even uptake of DNA molecules into multiple cells. The preculturing of cells has two benefits. It renders the cells more resistant to the electrical pulses, and increases the totipotency of such cells immediately prior to electroporation. This is in contrast to other methods, such as the biollistic method (see U.S. Pat. No. 9,945,050) which entails the bombardment of cells with metal particles coated with DNA. Such a procedure results in clustered and scattered transformation areas. Many cells are either not transformed or are fatally wounded by the impact of the particle.

It is important for the methods of the invention to carefully measure and modify the resistance of the electroporation buffer, and the pulse times. The method is useful for the transformation and generation of transgenic plants, including monocots, dicots, and gymnosperms.

In another embodiment of the present invention, an alternative method of creating marker-free transgenic plants involves the use of the isopentenyl transferase (IPT) gene. This embodiment relies on the ability of cells transformed with the IPT gene to trigger cell division and meristem formation in target cells and adjacent cells (H. J. Klee at al., Annu Rev. Plant Physiol 38, 467 (1987)).

The IPT gene is found on the Ti plasmid of Agrobacterium tumefaciens. The enzyme encoded by the IPT gene is described as a isopentenyltransferase capable of catalyzing the condensation of dimethylallyl-pyrophosphate (DMAPP) with adenosine 5′ monophosphate (AMP) to produce Zeatinriboside-5′ monophosphate (ZMP), a precursor of several cytokinins (Astot et al., Proc. Natl. Sci. USA, 97,14778 (2000).

Others have noted that plant cells transformed with a vector containing the IPT gene are induced to form shoots on hormone-free culture media (Ebinuma et al., In Vitro Cell. Dev. Biol., 37,103,(2001)). However, since the overproduction of cytokinin results in abnormal phenotypes, such plants are of little commercial value. They have further found that, subsequent to shoot regeneration, it is possible to remove the IPT gene from transformed cells by simultaneously introducing a recombinase enzyme in conjunction with the correct recognition sites surrounding the IPT gene. Such IPT-recombinase gene combinations can be used to generate marker free transgenic plants.

It has been observed by others that IPT transgenic cells can stimulate cell division in neighboring non-transgenic cells (Ebinuma et al., Plant Biotechnol. 14, 133 (1997)). However, in the method described herein, instead of producing non-transgenic cells, one may produce trait-gene-containing transgenic cells which do not contain the IPT gene. These trait-gene-containing cells may be induced to form shoots by the neighboring IPT-gene-containing cells. The method of this embodiment is directed to introducing the IPT gene and the desired trait genes on separate molecules (i.e., in trans). By introducing the DNA molecules in trans it is expected that, in certain instances, the trait gene will be introduced into cells adjacent to cells in which the IPT gene will be introduced. Moreover, by introducing the IPT gene in trans, there is no requirement for the use of a recombinase enzyme, or similar function, to remove said IPT gene from cells that “only” contain the desired trait gene, because the transgenic cells of interest already lack the IPT gene.

Thus, the method of this embodiment entails co-transforming plant tissue with a mixture of plasmids for IPT and a trait gene. Some cells will receive only the IPT gene, some only the trait gene, and some will take up both genes. In the case where cells containing only the IPT gene are adjacent to cells containing only the desired trait gene it is expected that the IPT gene containing cells will drive shoot formation in trait gene containing cells. Alternatively, since this method relies on co-transforming the IPT gene on a separate plasmid from the trait gene, it is also possible that both plasmids will be taken up by the same cell, however, just as in previous embodiments, the IPT gene may be only transiently expressed in the target cell during the first few days after transformation, and will thereafter be lost from the cell. Such transient expression may be sufficient to produce enough IPT within these neighboring cells to cause the cell division necessary for shoot formation in the transgenic cells.

The method of the invention can also be performed by using genes other than IPT that are capable of stimulating or modifying cell division and/or cell growth through the action of diffusible compounds whose production is directed by said gene product, herein termed “stimulatory” genes. For example, one skilled in the art could make use of a gene involved in the biosynthesis of other plant growth regulators, including but not limited to plant auxins, brassinosteriods, gibberelic acid, jasmonic acid, and ethylene.

When practicing the embodiments of the invention directed to cotransformation of IPT (or other cell division/growth stimulating genes), one may choose any of a variety of means known in the art for the introduction of the DNA to the cells, including but not limited to electroporation, agrobacterium-mediated transformation, the gene gun, immobilization of the DNA on silicon fibers, magnetophoretic transformation, and microinjection of the DNA.

Additional features of the invention will be apparent from the following illustrative Examples. All patents, publications, and other documents cited herein are hereby incorporated in their entirety.

### EXAMPLES

### Example 1

### Electroporation of Chrysanthemum Cultured Explants

Two different tissue culture stages of chrysanthemum tissue and two different media compositions were evaluated in this Example.

Plant Material. Chrysanthemum morifolium, Ramat var. Aspen (PP005240) plants were maintained in vitro on MS-B5 media (MS salts (Gibco), Sucrose (GibcoBRL #15503-022) 30 g/l, Casein Enzymatic Hydrolysate (Sigma # C-7290) 0.3 g/l, 1000× Gamborg B5 vitamins (Sigma # G-1019) 1 ml/l, Phytagel (Sigma # P-8169), 4 g/l, Indole Acetic Acid (Sigma) 0.1 mg/l.

Explant Preparation. Leaf explants (ca. 5 mm×5 mm) were excised from the central portion of the chrysanthemum leaves under sterile conditions. Explants were placed on IBD media (J.M. Sherman et al., J. Amer. Soc. Hort. Sci. 123,189(1998)). (MS salts, B5 vitamins, 0.1 g/l myso-inositol,0.23 mg/l BAP, 2 mg/l AA, 0.5 or 1 mg/12,4-D, 30 g/l sucrose, 4 g/l Phytagel) containing either 0.5 mg/l or 1 mg/l 2,4 D for either one or two weeks.

Electroporation. For each electroporation, ten cultured explants were placed in a 0.4 mm electroporation cuvette (Biorad Laboratories, IL, product number 165-2088) with 400 μl of electroporation buffer (10 mM Hepes, pH 5.6, 0.3M Mannitol), 20 μg (20 ul) of pFFK19, which contains the neomycinphosphotransferase (NPTII) gene conferring kanamycin resistance gene NPTII (Timmermans et al., J. Biotechnol. 14, 333 (1990)), and 50 μg (50 ul) of pWAC2 (An et al., Plant J. 1996:10, 107) containing the Arabidopsis thaliana Actin2 promoter linked the to the coding region of beta-glucuronidase (GUS) from E. coli. (Jefferson et al., EMBO J., 6, 3901 (1987)).

For each tissue culture stage, four different electroporation conditions were tested. Controls comprised cultured explants that were not electroporated and were not incubated with plasmid. After electroporation all cultured explants were transferred to IB media (MS salts, B5 vitamins, 0.1 g/l myo-inositol, 0.23 mg/l BAP, 2 mg/l IAA, 30 g/l sucrose, 4 g/l, Phytagel) for 2-4 days. Cultured explants were then transferred to IB media containing kanamycin (100 mg/l) for 4-6 weeks and then placed on rooting media containing (MS salts, 30% sucrose, 0.3/liter Casein Enzymatic hydrolysate, B5 vitamins, 0.4% Phytagel and 0.1 mg/liter NAA).

Co-transformation of NPTII and GUS genes. The number of cultured explants producing shoots on selective media for each electroporation condition was scored (Table 1). The transgenic nature of the shoots was confirmed by polymerase chain reaction assays using the following oligonucleotide primers to the kanamycin resistance gene contained in pFFK19.

KAN-F 5′ AGC TGT GCT CGA CGT TGT CAC 3′ [SEQ ID NO. 1]

KAN-R 5′ AAT CGG GAG CGG CGA TAC CG 3′ [SEQ ID NO. 2]

In addition, the frequency of plants containing both the NPTII gene and the GUS gene was determined by polymerase chain reaction assays using the following oligonucleotide primers to the GUS gene contained in pWAC2:

GUS-F 5′ CGT GGT GAT GTG GAG TAT TGC 3′ [SEQ ID NO. 3]

GUS-R 5′ TTG CAG CAG AAA AGC CGC C 3′ [SEQ ID NO. 4]

The number of shoots determined positive for each set of NPT and GUS primers is shown below in Table 1.

TABLE 1
Electroporation conditions, selection data, and PCR data for the transformation of
Chrysanthemum cultured explants. PCR data denotes the number of shoots showing a
positive signal with primers for the following genes: K, NPTII gene; G, GUS gene; KG,
NPTII and GUS. N is a designated experiment number; μF is Capacitance in
microFaradays; V, volts; kv/cm, Kilovolts per cm; R, resistance in ohms;
msec is duration of electroporation pulse.

| | | | | | | Selection Data | | | | | | |
|---|---|---|---|---|---|---|---|---|---|---|---|---|
| | | | | | | | Explants | | | | | |
| Electroporation Conditions | | | | | | | with | Shoots | | | | |
| | | | kv/ | | | Explant | shoots on | on | PCR data | | | |
| N | μF | V | cm | R | msec | number | Kan100 | Kan100 | K | G | KG | Total |

1 week on IBD (0:5 mg/l 24D)

| | | | | | | | | | | | | |
|---|---|---|---|---|---|---|---|---|---|---|---|---|
| 1 | 960 | 100 | 0.250 | 800 | 784 | 10 | 0 | 0 | 0 | 0 | 0 | 0 |
| 2 | 500 | 100 | 0.250 | 800 | 381 | 10 | 0 | 0 | 0 | 0 | 0 | 0 |
| 3 | 250 | 100 | 0.250 | 800 | 200 | 11 | 2 | 9 | 1 | 0 | 5 | 6 |
| 4 | 100 | 100 | 0.250 | 800 | 76 | 10 | 0 | 0 | 0 | 0 | 0 | 0 |

1 week on IBD (1 mg/l 24D)

| | | | | | | | | | | | | |
|---|---|---|---|---|---|---|---|---|---|---|---|---|
| 1 | 960 | 100 | 0.250 | 700 | 759 | 10 | 0 | 0 | 0 | 0 | 0 | 0 |
| 2 | 500 | 100 | 0.250 | 800 | 353 | 10 | 0 | 0 | 0 | 0 | 0 | 0 |
| 3 | 250 | 100 | 0.250 | 700 | 175 | 10 | 2 | 7 | 2 | 0 | 1 | 3 |
| 4 | 100 | 100 | 0.250 | 700 | 67 | 10 | 1 | 2 | 0 | 0 | 0 | 0 |

2 weeks on IBD (0.5 mg/l 24D)

| | | | | | | | | | | | | |
|---|---|---|---|---|---|---|---|---|---|---|---|---|
| 1 | 950 | 100 | 0.250 | 800 | 800 | 8 | 3 | 6 | 0 | 1 | 2 | 3 |
| 2 | 500 | 100 | 0.250 | 800 | 419 | 10 | 0 | 0 | 0 | 0 | 0 | 0 |
| 3 | 250 | 100 | 0.250 | 800 | 200 | 9 | 4 | 12 | 4 | 1 | 5 | 10 |
| 4 | 100 | 100 | 0.250 | 800 | 84 | 9 | 2 | 15 | 3 | 1 | 5 | 9 |

2 weeks on IBD (1 mg/l 24D)

| | | | | | | | | | | | | |
|---|---|---|---|---|---|---|---|---|---|---|---|---|
| 1 | 950 | 100 | 0.250 | 700 | 707 | 10 | 0 | 0 | 0 | 0 | 0 | 0 |
| 2 | 500 | 100 | 0.250 | 800 | 354 | 10 | 0 | 0 | 0 | 0 | 0 | 0 |
| 3 | 250 | 100 | 0.250 | 800 | 178 | 10 | 0 | 0 | 0 | 0 | 0 | 0 |
| 4 | 100 | 100 | 0.250 | 800 | 81 | 10 | 2 | 15 | 1 | 2 | 9 | 12 |

| | |
|---|---|
| Total number of shoots selected on Kan 100 mg/l (S+ve) | 66 |
| Total Number of PCR Kan positives (K+ve) | 38 |

| | | | | |
|---|---|---|---|---|
| Total number of transgenic shoots for each gene (T) | 11 | 5 | 27 | 43 |

| | |
|---|---|
| Selection Efficiency. (K+ve/S+ve) | 58% |
| Escape percentage 1 − (K+ve/S+ve) | 42% |
| Marker free efficiency (G/T) | 12% |

| | | | | |
|---|---|---|---|---|
| Co-transformation frequency as a percentage of NPTII(K) positives | 29% | — | 71% | 100% |
| Co-transformation frequency as a percentage of total transgenics | 26% | 11% | 63% | 100% |

The data in Table 1 shows that 58% of plants selected on kanamycin media contained the NPTII gene. Furthermore, a high percentage (71%) of plants containing the NPTII gene also contained the GUS gene. Although others have reported the cotransformation of unlinked genes into protoplasts by electroporation (Christou et al., Theor Appl Genet 79, 337 (1990)), Schocher et al., Biotechnology 4,1093 (1986)), and others have reported the cotransformation of unlinked genes into intact cells by use of the gene gun (Wakita et al., Genes Genet. Syst. 73, 219 (1998)), this is the first description of co-transformation of unlinked genes into intact cells by electroporation.

The level of activity of the GUS gene was assayed by measuring the conversion of 4-Methylumbelliferyl β-D-Glucuronide (XMUG) (Sigma M5664) by protein extracts of the transgenic plants (Jefferson et al., EMBO J., 6, 3901 (1987)). Table 2 below shows the relative level of GUS activity in lines shown to have a GUS insert.

TABLE 2
Relative GUS activity in transgenic lines with GUS insert.
WT control, non-transgenic wild type control plant.
Transgenic lines are coded according to the culture
and electroporation conditions used. 1W, 1 week;
2W, 2 weeks; 0.5, 0.5 mg/l 2,4-D; 1.0, 1 mg/l 2,4-D.
Subsequent numbers denote electroporation condition (1-4),
explant number (1-10), and shoot number from
each explant. Lines with only GUS gene and no NPT
gene are denoted as ‘+’.

| Line Number | GUS activity | Line Number | GUS activity |
|---|---|---|---|
| 1W-0.5-3-1-2 | 194 | 2W-0.5-4-1-5 | 36.3 |
| 1W-0.5-3-1-3 | 344 | 2W-0.5-4-1-6 | 31.1 |
| 1W-0.5-3-1-5 | 318 | 2W-0.5-4-1-7 | 96 |
| 1W-0.5-3-1-7 | 389 | 2W-0.5-4-2-6 | 14 |
| 1W-0.5-3-1-8 | 101 | 2W-0.5-4-2-7+ | 23.1 |
| 1W-1.0-3-1-3 | 17 | 2W-0.5-4-2-8 | 49 |
| 2W-0.5-1-1-2 | −49 | 2W-1.0-4-1-1 | 68 |
| 2W-0.5-1-3-1 | 559 | 2W-1.0-4-1-2+ | 70 |
| 2W-0.5-1-3-3+ | 115 | 2W-1.0-4-1-3+ | 142.3 |
| 2W-0.5-3-1-2 | 267 | 2W-1.0-4-2-1 | 38.9 |
| 2W-0.5-3-1-4 | 21.6 | 2W-1.0-4-2-2 | 52.5 |
| 2W-0.5-3-2-2 | 95.5 | 2W-1.0-4-2-3 | 58 |
| 2W-0.5-3-2-3 | 578 | 2W-1.0-4-2-4 | 15.2 |
| 2W-0.5-3-2-4 | 97 | 2W-1.0-4-3-2 | 103 |
| 2W-0.5-3-2-5+ | 44 | 2W-1.0-4-3-5 | 191 |
| | | 2W-1.0-4-3-6 | 118 |
| WT Control | 9 | 2W-1.0-4-4-2 | 55 |

All transgenic plant lines show significant GUS activity relative to the wild type non-transgenic line. Wild-type “control” had a value of 9, while transgenics had values ranging from 14 to 559. As expected, considerable variation in GUS activity is seen. This is most likely due to “position effect” exerted by flanking genomic sequences into which the GUS gene has integrated. This is a phenomenon well known to those skilled in art. In practice, this variation indicates the need to initially screen many independent transformants, to identify those with the most suitable level of expression.

Optimal Conditions. Table 3 below summarizes the number of transgenic shoots obtained for each electroporation condition used.

TABLE 3
Average pulse time and total number of transgenic
shoots obtained.

| | | | | | | Total | Explants | |
|---|---|---|---|---|---|---|---|---|
| | | | | | Average | number | with | PCR |
| | | | kv/ | | Time | of leaf | shoots on | Positive |
| N | μF | V | cm | R | msecs | discs | Kan 100 | shoots |
| 1 | 950 | 100 | 0.250 | 700 | 603 | 38 | 3 | 3 |
| 2 | 500 | 100 | 0.250 | 800 | 377 | 40 | 0 | 0 |
| 3 | 250 | 100 | 0.250 | 800 | 188 | 39 | 8 | 22 |
| 4 | 100 | 100 | 0.250 | 800 | 77 | 39 | 5 | 21 |

The most efficient level of transformation was found using 100 Volts with a capacitance of about 250 μF yielding a pulse of time of about 190 milliseconds. The exact capacitance needed to produce the preferred pulse length will vary, depending on the exact resistance of the final buffer and callus mixture.

Marker-Free Transgenic Plants (Method I)

The PCR data shown in Table 1 indicate that 58% of all plants selected on kanamycin media contained the NPTII gene. Conversely, 42% of all plants selected on kanamycin media contained no NPTII gene. Such false positives plants are known as “escapes” which escape selection by kanamycin and are ordinarily presumed to be normal wild-type plants. However, among the kanamycin escapes in this Example, five plants (12% of all transgenic plants) contained the GUS gene. These plants are transgenic plants that do not contain a selectable marker; i.e., they are marker-free transgenic plants.

### Example 2

### Electroporation of Multiple Genes into Chrysanthemum

In the previous Example, selectable marker and trait genes were transformed into chrysanthemum tissue on separate plasmids. In this example, three independent genes on three independent molecules were electroporated into intact cultured chrysanthemum explants. The ability to “stack” multiple traits in a transgenic plant is of significant commercial value. Here, the gai gene controls plant height (Peng et al., Nature, 400, 261, (1999)), the CONSTANS gene (CO) controls flowering time (Putterill, J, et al,. Cell, 80, 847, (1995.), and the third gene, the plasmid p4161, is used as a selectable marker.

Construction of p4161. Plasmid p4161 contains the Ubiquitin 3 promoter from Arabidopsis thaliana var landsberg linked to the NPTII gene with the NOS terminator. The Ubiquitin promoter was cloned from Arabidopsis thaliana var landsberg genomic DNA by PCR using primers designed using published DNA sequence information (S.R. Norris et al., Plant Mol. Biol 21, 895 (1993) Gene bank accession # L05363) as shown below:

[SEQ ID NO:5]

| | |
|---|---|
| Ubi3-F: | 5′GGA AAG CTTCGG ATT TGG AGC 3′ |
| | HinDIII |

[SEQ ID NO:6]

| | |
|---|---|
| Ubi3-R: | 5′CGG CTG CAGCGT CTG AAA TAA AAC AAT AGA AC 3′ |
| | PstI |

The resulting 1752 bp fragment was digested with PstI and HindII and cloned into the HindIII and Pst sites of pUC19 to create a pUC-Ubi3 plasmid. The NTPII coding region was PCR amplified from pFF19K using the following primers:

[SEQ ID NO:7]

| | |
|---|---|
| NTP-F: | 5′TGA GGA TCCTTT CGC ATG ATT G 3′ |
| | BamHI |

[SEQ ID NO:8]

| | |
|---|---|
| NTP-R: | 5′TTG GTA CCC CAG AGT CCC GC 3′ |
| | KpnI |

The resulting 819 bp fragment was digested with BamH1 and Kpn1 and ligated into the pUC-Ubi3 plasmid to create a pUCUbi3-Km plasmid. The plasmid pWAC2 was digested with EcorRI Sac and a 271 bp fragment containing the NOS terminator was introduced into the pUC-Ubi3-Km vector to create p4161.

50 μg of p4161, 50 μg of plasmid λg (courtesy of Nicholas P. Harberd, John Innes Centre, Colney Lane, Norwich, England) containing a 5 kb insert containing the genomic gai gene from Arabidopsis thaliana (Peng et al., Nature, 400, 261, (1999)), and 50 μg of the plasmid g39 (courtesy of George Coupland, John Innes Centre, Norwich, England) containing the entire CONSTANS gene from Arabidopsis thaliana (Putterill, J, et al,. Cell, 80, 847, (1995)), were dissolved in electroporation buffer (as described in Example 1) and added to a 0.4 cm electroporation cuvette as described herein. Two different electroporation conditions were examined (with duplicates of each). Condition E-1 comprised 50 μF at 100V with a pulse time of 119 and 132 milliseconds. Condition E-2 comprised 100 μF with pulse times of 193 and 208 milliseconds. Ten calli were used for each electroporation. After electroporation, calli were placed on IBD for two days, and then transferred to IB with 50 μg/ml kanamycin for 1 month to allow for selection of kanamycin positive shoots. The conditions are summarized in Table 3 below.

TABLE 4
Electroporation conditions for the transfer of three independent
plasmids into cultured chrysanthemum explants.

| | | V | E | | |
|---|---|---|---|---|---|
| N | C μF | Volt | kv/cm | R | Time |
| E-1-1 | 50 | 100 | 0.250 | 800 | 132 |
| E-1-2 | 50 | 100 | 0.250 | 800 | 119 |
| E-2-1 | 100 | 100 | 0.250 | 800 | 193 |
| E-2-2 | 100 | 100 | 0.250 | 800 | 208 |

After two months, kanamycin positive shoots are screened by PCR for the presence of the NPTII gene, the gai gene and the CO gene. It is expected that some plants will contain all three genes, some plants will have two genes present, and some plants will have only one of the genes present. It is also expected that escapes will be produced which either or both of the gai and CO genes.

This method allows for the insertion of multiple genes into a given plant species, without the need for multiple transformation events, and/or cross-hybridization.

### Example 3

### Transient Expression in Petunia

In this Example, a method of the invention is demonstrated in petunia, a commercially important dicot genus. Explants consisting of the uppermost young leaves of petunia plants (Petunia integrifolia) were excised and surface sterilized using 10% bleach. After washing, leaves were cut into about 10×10 mm leaf-discs and placed on Petunia Callus Induction Media (PCI media; MS Salts, B5 vitamins, 30 g/l sucrose, Ph5.8, 4 g/l Phytagel, 1 mg/l BAP, 0.1 mg/l NAA, 2 mg/l 2,4-D) and cultured in the dark for 25-30 days. The resulting callus was sub-cultured on NAS medium for one month (NAS media is composed of: Chu-N6 Salts (Sigma C1416)), B5 vitamins (Sigma G1019), 0.3 g/l casein enzymatic hydrolysate, 30 g/l sucrose, 10 g/l D-Sorbitol), 1 mg/l 2,4-D, 0.1 mg/l kinetin (Sigma K0753), 0.2 mg/l IAA (Sigma I2886), pH 5.7, 4 g/l Phytagel (Sigma # P8169). Compact regenerable type I callus was sub-cultured to select for friable, fast growing type II callus, for sub-culturing on the same medium.

Prior to electroporation, calli were incubated on ice for 30 minutes in electroporation buffer (10 mM Hepes, pH 5.6, 0.3M Mannitol) containing 20 μg of pWAC2 plasmid DNA in 0.4 cm electroporation cuvettes.

Approximately 15 calli, each measuring about 3 mm×3 mm were used for each electroporation. Calli were subjected to a single pulse of ranging from 0.25 to 1.25 kV/cm and capacitance ranging from 250 to 1000 μF. After two days incubation in liquid NAS growth media the level of activity of the GUS gene was assayed by measuring the conversion of 4-Methylumbelliferyl B-D-Glucuronide (XMUG) (Sigma M5664) by protein extracts of transformed calli described by in Jefferson et al., EMBO J., 6, 3901 (1987). The results are shown in Table 5 below.

TABLE 5
Transient expression of the GUS gene in Petunia integrifolia calli.
C−, control without DNA and no electroporation. C+,
control with DNA and no electroporation.

| N | μF | V | kv/cm | R | msec | GUS |
|---|---|---|---|---|---|---|
| 1 | 960 | 100 | 0.250 | 400 | 368 | 33 |
| 2 | 960 | 250 | 0.625 | 500 | 345 | 42 |
| 3 | 960 | 500 | 1.250 | 500 | 193 | 56 |
| 4 | 500 | 100 | 0.250 | 300 | 157 | 25 |
| 5 | 500 | 250 | 0.625 | 400 | 147 | 25 |
| 6 | 500 | 500 | 1.250 | 500 | 133 | 49 |
| 7 | 250 | 100 | 0.250 | 500 | 123 | 74 |
| 8 | 250 | 250 | 0.625 | 400 | 76.4 | 67.4 |
| 9 | 250 | 500 | 1.250 | 400 | 67.5 | 93.6 |
| C− | | | | | | 6.1 |
| C+ | | | | | | 7.6 |

The highest expression level was detected in calli subjected to the mild electroporation conditions (250 μF and 0.250 to 1.250 kV/cm) with the highest expression level at 1.250 kV/cm with pulse times in the range of 67.5 to 368 msecs.

### Example 4

### Stable Transformation of Petunia

Fifteen calli measuring approximately 3 mm×3 mm were placed in 400 μl of electroporation with 100 μg of pWAC2 and 100 μg pFF19K. The mixture was allowed to stand on ice for 30 minutes. After electroporation calli were placed on BNI medium for two days and then transferred to BNI containing 100 mg/l Kanamycin.

Twelve different electroporation conditions were tested (conditions 1-12). Calli were subjected to single pulses of field strength ranging from 0.1 to 0.5V/cm with a capacitance ranging from 100 to 975 μF, with pulse times from 22 to 217 msec. Controls examined the growth of callus on selective and non-selective media in the presence and absence of DNA without electroporation Neither control (with and without DNA grew) on selective media. The number of shoots appearing on BNI plus 100 mg/l kanamycin was scored for each of the 12 experimental conditions. Shoots were then transferred to rooting media (BNRT2; MS salts, B5 Vitamins, 0.3 g/l casein enzymatic hydrolysate, 40 g/l sucrose, 0.5 mg/1 IBA, pH 5.7, 4 g Phytagel plus 100 mg/l Kanamycin) and the number of rooted shoots scored. The results are shown below in Table 6.

TABLE 6
Electroporation conditions and plant selection data for
petunia callus electroporated with pWAC2 and pFF19K.

| | | | | | | | Rooted |
|---|---|---|---|---|---|---|---|
| | | | | | | Shoots on | Shoots |
| | | | | | | BNI Kan | BNRT2 Kan |
| N | μF | V | kv/cm | R | msec | 100 mg/l | 100 mg/l |
| 1 | 960 | 100 | 0.250 | 200 | 217 | 62 | 24 |
| 2 | 960 | 250 | 0.625 | 300 | 197 | 0 | 0 |
| 3 | 960 | 500 | 1.250 | 300 | 152 | 0 | 0 |
| 4 | 500 | 100 | 0.250 | 200 | 102 | 0 | 0 |
| 5 | 500 | 250 | 0.625 | 200 | 100 | 0 | 0 |
| 6 | 500 | 500 | 1.250 | 200 | 79 | 0 | 0 |
| 7 | 250 | 100 | 0.250 | 200 | 56 | 67 | 16 |
| 8 | 250 | 250 | 0.625 | 200 | 55 | 0 | 0 |
| 9 | 250 | 500 | 1.250 | 200 | 43 | 0 | 0 |
| 10 | 100 | 100 | 0.250 | 300 | 28 | 9 | 3 |
| 11 | 100 | 250 | 0.625 | 200 | 21 | 0 | 0 |
| 12 | 100 | 500 | 1.250 | 200 | 20 | 0 | 0 |

A relatively high number of shoots were seen in conditions 1, 7, and 10. Many of these appeared to be escapes since they failed to produce roots on rooting media containing kanamycin. The highest number of rooted shoots were seen using condition 1. Condition 1 (100 volts and 217 msecs) produced 24 rooted shoots. Condition 7 (100 volts, 56 msecs) produced 16 rooted shoots and condition 10 (100 volts 28 msecs) produced 3 rooted shoots. All other conditions failed to give any shoots on BNI medium.

For stable transformation of Petunia integrisola, it appears that the optimum conditions comprise an impulse time of about 200 msec together with mild electroporation parameters, such as 100 volts at 0.25 kv/cm. Although more severe conditions and shorter pulse times can be used to drive transient expression, they do not appear to result in the production of high numbers of stable transformants. Higher voltages presumably interfere with regenerative ability of petunia cells, by causing excessive cell damage or death.

### Example 6

### Marker-Free Transgenic Plants Using IPT Gene Method (Method II)

The transformation methods of the invention were applied to a representative woody genus (Rosa) using a positive selectable marker that, as defined herein functions as a “stimulatory gene” allows for the selection of marker free transgenic plants. The positive selectable marker used was the IPT gene which, as described herein, encodes and enzyme involved in a key step in cytokinin biosynthesis.

Isolation of IPT gene. The IPT gene was cloned from Agrobacterium tumefaciens C58 (American Type Culture Collection, item # 33970) by PCR using the following primers:

IPT-F 5′ TGT GGC ATT TAT TGA AAT GGC ACT G [SEQ ID NO: 9]

IPT-R 3′ CTA TAT CTA GAC ATC GTA ATT TTA AGA CG [SEQ ID NO: 10]

These primers were designed using published DNA sequence information (Barker et al, Plant Mol. Biol. 2, 335(1983) National Center for Biotechnology Information accession number NC-2377). The primers are used to amplify the region −501 to +1486 (relative to the ATG start of translation) of the IPT gene. The 1.9 kb fragment was blunt end ligated into the Smal site of pSP72 (Promega Corporation ((Madison Wis.) NCBI accession #X65332)) to create pIPT, and its identity confirmed by sequencing.

Petiole segments (about 5 mm in length) of Rosa hybrida var Bucbi, Carefree Beauty (U.S. Plant Pat. No. 4225), were cultured on Rose Callus Induction media (RCI) (MS salts, B5 vitamins, 2,4-D 3 mg/l, Kinetin 0.3 mg/l, 50 uM silver nitrate) in the dark for 2 and 5 weeks. For each electroporation condition tested, ten calli were placed in a 0.4 mm cuvette containing 400 μl of electroporation buffer (as described in Example 1 with 20 μg of pIPT. The mixture was allowed to stand on ice for 30 minutes. The 2 and 5 week calli were subjected to four different electroporation conditions as detailed below in Table 7.

TABLE 7
Electroporation conditions for cultured rose explants with pIPT.
1a-4a, explants cultured for 2 weeks. 1b-4b, explants
cultured for 5 weeks.

| N | μF | V | kv/cm | R | msec |
|---|---|---|---|---|---|
| 1a | 975 | 100 | 0.250 | 700 | 564 |
| 2a | 500 | 100 | 0.250 | 800 | 377 |
| 3a | 250 | 100 | 0.250 | 800 | 238 |
| 4a | 100 | 100 | 0.250 | 800 | 111 |
| 1b | 975 | 100 | 0.250 | 500 | 408 |
| 2b | 500 | 100 | 0.250 | 700 | 317 |
| 3b | 250 | 100 | 0.250 | 800 | 179 |
| 4b | 100 | 100 | 0.250 | 700 | 68 |

After electroporation, explants were placed on MS media containing no selective agent and no plant hormones. Control explants were also placed on MS media. Embryogenic structures that only appeared in Experiment 3a (100 volts, 238 msecs for 2 week calli) indicated that the IPT gene has been successfully introduced into the explants under the conditions used.

The method described in this Example can be combined with those in the previous examples to introduce one or more additional genes encoding desired traits. Based on the ability of the IPT gene to cause shoot formation in transformed as well as in adjacent non-IPT transformed cells, it is expected that shoots would appear that would contain the trait gene yet would not contain the IPT gene. Such trait-gene containing shoots lacking the IPT gene can readily be distinguished from IPT gene containing shoots by PCR using primers IPT-F and ITP-R described herein, and primers for the desired trait gene.

We claim:

1. A method for transforming a plant with a transgene, comprising the steps of:
a. culturing an intact explant of the plant in nutritive medium;
b. electroporating the explant with a pulse length of at least about 50 milliseconds to produce a transformed explant;
wherein the transgene is stably integrated into a chromosome of a cell of the transformed explant.

2. The method of claim 1, wherein the pulse length is from about 90 to about 300 milliseconds.

3. The method of claim 1, wherein the pulse length is from about 90 to about 250 milliseconds.

4. The method of claim 1, wherein the pulse length is from about 90 to about 200 milliseconds.

5. The method of claim 1, wherein the pulse length is from about 90 to about 150 milliseconds.

6. The method of claim 1, wherein at least two transgenes are electroporated in step b.

7. The method of claim 1, wherein a marker gene is also electroporated in step b.

8. The method of claim 6, wherein a marker gene on a separate DNA molecule is also electroporated in step b.

9. A method of producing a transgenic plant comprising the steps of:
a. culturing an intact explant of a plant in nutritive medium;
b. electroporating the explant with a pulse length of from about 50 to about 500 milliseconds to produce a transformed explant, wherein the transgene is stably integrated into a chromosome of a cell of the transformed explant; and
c. regenerating the transgenic plant from said transformed explant.

10. The method of claim 9, wherein the pulse length is from about 90 to about 300 milliseconds.

11. The method of claim 9, wherein the pulse length is from about 90 to about 250 milliseconds.

12. The method of claim 9, wherein the pulse length is from about 90 to about 200 milliseconds.

13. The method of claim 9, wherein the pulse length is from about 90 to about 150 milliseconds.

14. The method of claim 9, wherein at least two transgenes are electroporated in step b.

15. The method of claim 9, wherein a marker gene is also electroporated in step b.

16. The method of claim 9, wherein a marker gene on a separate DNA molecule is also electroporated in step b.

17. The method of claim 16, wherein the transgenic plant lacks the marker gene.

18. The method of claim 16, wherein the marker gene is the IPT gene.

19. The method of any of claims 1-18 wherein the plant is selected from the group consisting of monocots, dicots, and gymnosperms.

20. The method of claim 19 wherein the plant is selected from the group consisting of chrysanthemum, petunia, and rose.

21. A transgenic plant produced by the method of any of claims 1-18.

22. A transgenic plant produced by the method of claim 19.

23. A transgenic plant produced by the method of claim 20.

24. A method of producing a transgenic plant lacking a marker gene, comprising the steps of:
a. culturing intact plant tissue;
b. transforming the plant tissue with a transgene and a stimulatory gene, wherein the trait gene and the stimulatory gene are on separate nucleic acid molecules, to produce transformed plant tissue, wherein the transgene is stably integrated into a chromosome of a cell of the transformed plant tissue, and wherein the stimulatory gene is present in at least one cell of the plant tissue;
c. regenerating transgenic plants from said transformed plant tissue; and
d. selecting transgenic plants which lack the stimulatory gene.

25. The method of claim 24, wherein the transformation of step b. is performed by a method selected from the group consisting of agrobacterium-mediated transformation, the gene gun, magnetophoretic delivery, immobilization of the nucleic acids on silicon fibers, and microinjection of nucleic acids.

26. The method of claim 24, wherein the stimulatory gene is selected from the group consisting of IPT and genes involved in the biosynthesis plant growth regulators.

27. The method of claim 26, wherein the stimulatory gene is IPT.
